(19) United States
(12) Patent Application Publication
(10) Pub. No.: US 2009/0263338 A1
(43) Pub. Date: Oct. 22, 2009

(54) NAIL VARNISH

(75) Inventors: Nicolas ROLLAND, Asnieres Sur Seine (FR); Laurence GUERCHET, Chevilly La Rue (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/423,834

(22) Filed: Apr. 15, 2009

Related U.S. Application Data
(60) Provisional application No. 61/056,114, filed on May 27, 2008.

(30) Foreign Application Priority Data
Apr. 18, 2008 (FR) 08 52654

(51) Int. Cl. A61K 8/81; A61Q 3/02

(52) U.S. Cl. 424/61

(57) ABSTRACT

Nail varnish composition in the form of an emulsion containing one or more water-soluble or water-dispersible polymers obtained from acrylamido-2-methylpropanesulphonic acid (or AMPS) or from one of its salts and from water-soluble monomers having ethylenic unsaturation(s) as gelling agent. Makeup method employing the composition. Dried films of the nail varnish.

### REFERENCE TO PRIOR APPLICATIONS

This application claims priority to U.S. provisional application 61/056,114, filed May 27, 2008; and to French patent application 08 52654, filed Apr. 18, 2008, both incorporated herein by reference.

### FIELD OF THE INVENTION

The present invention relates to an aqueous nail varnish composition in emulsion form, comprising at least one gelling agent as defined below, and to a method for making up the nails employing the composition. Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Generally, nails and false nails are made up using liquid makeup compositions, also commonly referred to as nail varnishes. This nail varnish is generally applied in the form of superimposed layers at the surface of the nail to be made up, an intermediate drying stage being observed between each layer of varnish applied. In fact, this makeup method does not prove to be completely satisfactory.

First of all, the application of the varnish requires a certain time. Furthermore, this type of makeup involves being repeated very soon due to its inadequate hold. This is because the varnish applied flakes off and its gloss decreases very rapidly, generally after three to five days. It is then necessary to carry out a makeup-removing stage and a fresh makeup operation.

Furthermore, conventional nail varnishes generally involve the use of volatile solvents which generates a disagreeable smell when applied.

Several alternatives have already been provided in attempting to overcome, at least in part, the abovementioned disadvantages. Thus, products for making up the nails have been provided in the form of a kit of two liquid nail varnish compositions. However, the improvement in the hold is in this case acquired at the expense of the application conditions, which double the number of layers to be applied.

Another alternative has consisted in developing nail varnish compositions based on the dispersion of film-forming polymers in an aqueous phase, which are therefore satisfactory in olfactory terms. Unfortunately, the corresponding varnishes turn out to exhibit an unsatisfactory hold over time.

Finally, the great majority of conventional nail varnishes are anhydrous and thus do not make possible the use of water-soluble compounds, such as water-soluble active principles.

### SUMMARY OF THE INVENTION

The inventors have found that, by preparing a nail varnish in the form of an emulsion comprising at least one specific gelling agent as described below, it was possible to obtain easy and rapid application, the formation of a homogeneous, glossy, adherent and flexible dry film and a reduced content of organic solvent(s). This emulsion also exhibits the advantage of being stable and fluid.

### DETAILED DESCRIPTION OF THE PREFERRED EMBODIMENTS

A subject-matter of the present invention is a nail varnish composition in the form of an emulsion, comprising one or more gelling agents as defined below.

A further subject-matter of the invention is a method for making up the nails employing the composition.

A further subject-matter of the invention is a dried nail varnish film obtained by drying the invention nail varnish composition on a substrate, such as a fingernail or other surface. Drying can be accomplished, for example, statically, for example by simple evaporation of volatile components at room temperature, and/or may be assisted by directing an optionally warmed fluid, like warm air, at the composition. Vacuum drying can also be used.

Other subject-matters, characteristics, aspects and advantages of the invention will become even more clearly apparent on reading the description and example which follow.

The nail varnish composition in the form of an emulsion comprises one or more water-soluble or water-dispersible polymers obtained from acrylamido-2-methylpropanesulphonic acid (or AMPS) or from one of its salts and from water-soluble monomers having ethylenic unsaturation(s) as gelling agent(s).

The term “nail varnish” is understood to mean a composition capable of forming a film on the nail.

The composition according to the invention is provided in the form of an emulsion; it thus comprises at least one continuous phase and at least one dispersed phase.

Preferably, the continuous phase predominantly comprises one or more oils (the reference is then to an oily continuous phase) or one or more organic solvents (the reference is then to an organic continuous phase) and the dispersed phase is aqueous.

The continuous phase preferably predominantly comprises one or more oils or one or more organic solvents, that is to say an amount of oil(s) and/or of organic solvent(s) preferably ranging from 40 to 99.99% by weight, better still from 60 to 99.99% by weight, with respect to the total weight of the continuous phase.

In particular, the continuous phase comprises at least one organic solvent and the composition is provided in the form of a water-in-organic solvent emulsion. Such an emulsion makes it possible, by the presence of a dispersed aqueous phase, to incorporate water-soluble active principles.

Preferably, the composition comprises an amount of continuous phase ranging from 10 to 85% by weight, better still from 30 to 80% by weight, with respect to the total weight of the composition. Preferably, the composition comprises an amount of dispersed phase ranging from 15 to 90% by weight, better still from 20 to 70% by weight, with respect to the total weight of the composition.

Preferably, the continuous phase and the dispersed phase of the emulsion are present in a ratio by weight ranging from 1/10 to 6 and better still from 2/5 to 4.

Mention may in particular be made, as examples of gelling agent which can be used in the composition according to the invention, of:
copolymers obtained from AMPS or from one of its salts and from acrylamide or from methacrylamide, such as, for example, the crosslinked acrylamide/sodium acrylamido-2-methylpropane-sulphonate copolymer as an emulsion in C₁₃-C₁₄ isoparaffin and laureth-7 (CTFA name: Polyacrylamide/C₁₃-C₁₄ Isoparaffin/Laureth-7) sold under the name Sepigel 305 by SEPPIC or the crosslinked acrylamide/sodium acrylamido-2-methylpropanesulphonate copolymer as a 40% inverse emulsion in isohexadecane and polysorbate-80 (CTFA name: Acrylamide/Sodium Acryloyldimethyltaurate/Isohexadecane/Polysorbate-80) sold under the name Simulgel 600 by SEPPIC;
copolymers of AMPS or of one of its salts and of vinylpyrrolidone or of vinylformamide, such as the products Ammonium Acryloyldimethyltaurate/VP Copolymer (INCI name) sold under the name Aristoflex AVC by Clariant;
copolymers of AMPS or of one of its salts and of sodium acrylate, such as, for example, the crosslinked AMPS/sodium acrylate copolymer as an inverse emulsion in a water/isohexadecane/sorbitan oleate mixture (CTFA name: Acrylamide/Sodium Acryloyldimethyltaurate/Isohexadecane/Polysorbate-80) sold under the name Simulgel EG by SEPPIC; and
copolymers of AMPS or of one of its salts and of hydroxyethyl acrylate, such as, for example, the crosslinked AMPS/hydroxyethylacrylate copolymer as an inverse emulsion in a polysorbate 60/squalane mixture (sold under the name Simulgel NS by SEPPIC).

More particularly, the gelling agent is chosen from copolymers obtained from AMPS or from one of its salts and from acrylamide or methacrylamide.

The gelling agent or agents are present in an amount preferably ranging from 0.01 to 10% by weight, better still from 0.01 to 5% by weight, with respect to the total weight of the composition according to the invention.

Preferably, the gelling agent or agents is or are present in the aqueous dispersed phase. It (they) is or are present in an amount preferably ranging from 0.01 to 35% by weight, better still from 0.01 to 25% by weight, with respect to the total weight of the dispersed phase.

The composition according to the invention preferably comprises one or more film-forming polymers. Advantageously, it is present in the continuous phase.

The term “film-forming polymer” is understood to mean, within the meaning of the present patent application, a polymer capable of forming, by itself alone or in the presence of an additional agent which is able to form a film, a continuous film on a support at a temperature ranging from 10° C. to 150° C.

The polymer can be dissolved or dispersed in the form of particles in the continuous phase.

Mention may be made, as film-forming polymers which can be used in the composition of the present invention, of synthetic film-forming polymers obtained by the radical route or by polycondensation, polymers of natural origin and their blends. Generally, these polymers can be random polymers, block copolymers of A-B, A-B-A multiblock or also ABCD type, or grafted polymers.

The term “synthetic polymer obtained by the radical route” (or radical polymer) is understood to mean a polymer obtained by polymerization of monomers having unsaturation(s) in particular ethylenic unsaturation(s), each monomer being capable of homopolymerizing (unlike polycondensates).

The radical film-forming polymers can in particular be acrylic and/or vinyl homopolymers or copolymers.

The vinyl film-forming polymers can result from the polymerization of monomers having ethylenic unsaturation(s) comprising at least one acid group and/or of the esters of these acidic monomers and/or of the amides of these acidic monomers.

Use may be made, as monomers having ethylenic unsaturation(s) comprising at least one acid group or monomer carrying an acid group, of α,β-ethylenic unsaturated carboxylic acids, such as acrylic acid, methacrylic acid, crotonic acid, maleic acid and itaconic acid. Use is preferably made of (meth)acrylic acid and crotonic acid and more particularly of (meth)acrylic acid.

The esters of acidic monomers are advantageously chosen from esters of (meth)acrylic acid (also known as (meth)acrylates), in particular alkyl (meth)acrylates, especially C₁-C₂₀ alkyl (meth)acrylates, more especially C₁-C₈ alkyl (meth)acrylates, aryl (meth)acrylates, in particular C₆-C₁₀ aryl (meth)acrylates, or hydroxyalkyl (meth)acrylates, in particular C₂-C₆ hydroxyalkyl (meth)acrylates.

Mention may be made, among alkyl (meth)acrylates, of methyl methacrylate, ethyl methacrylate, butyl methacrylate, isobutyl methacrylate, 2-ethylhexyl methacrylate and lauryl methacrylate.

Mention may be made, among hydroxyalkyl (meth)acrylates, of hydroxyethyl acrylate, 2-hydroxypropyl acrylate, hydroxyethyl methacrylate and 2-hydroxypropyl methacrylate.

Mention may be made, among aryl (meth)acrylates, of benzyl acrylate and phenyl acrylate.

Preferably, the esters of (meth)acrylic acid are alkyl (meth)acrylates.

According to the present invention, the alkyl group of the esters can be either fluorinated or perfluorinated, that is to say that a portion or all of the hydrogen atoms of the alkyl group are replaced by fluorine atoms.

Mention may be made, as amides of the acidic monomers, for example, of (meth)acrylamides, in particular N—(C₂-C₁₂ alkyl)(meth)acrylamides. Mention may be made, among N—(C₂-C₁₂ alkyl)(meth)acrylamides, of N-ethylacrylamide, N-(t-butyl)acrylamide and N-(t-octyl)acrylamide.

The vinyl radical film-forming polymers can also result from the homopolymerization or from the copolymerization of monomers chosen from vinyl esters and styrene monomers. In particular, these monomers can be polymerized with acidic monomers and/or their esters and/or their amides, such as those mentioned above.

Mention may be made, as examples of vinyl ester, of vinyl acetate, vinyl neodecanoate, vinyl pivalate, vinyl benzoate and vinyl t-butyl benzoate.

Mention may in particular be made, as styrene monomers, of styrene and α-methylstyrene.

The list of the monomers which is given is not limiting and it is possible to use any monomer known to a person skilled in the art coming within the categories of acrylic and vinyl monomers (including the monomers modified by a silicone chain).

Use may also be made, as vinyl polymer, of silicone-acrylic polymers.

Mention may also be made of the polymers resulting from the radical polymerization of one or more radical monomers inside and/or partially at the surface of preexisting particles of at least one polymer chosen from the group consisting of polyurethanes, polyureas, polyesters, polyesteramides and/or alkyd resins. These polymers are generally referred to as “hybrid polymers”.

Mention may be made, as film-forming polymer obtained by polycondensation (or film-forming polycondensate), of polyurethanes, which may be anionic, cationic, nonionic or amphoteric, polyurethane-acrylics, polyurethane-polyvinyl-pyrrolidones, polyester-polyurethanes, polyether-polyurethanes, polyureas, polyurea/polyurethanes, silicone polyurethanes and their blends.

The film-forming polyurethane can, for example, be an aliphatic, cycloaliphatic or aromatic polyurethane, polyurea/urethane or polyurea copolymer comprising, alone or as a mixture, at least one block chosen from:
a block of aliphatic and/or cycloaliphatic and/or aromatic polyester origin, and/or
a branched or unbranched silicone block, for example a polydimethylsiloxane or polymethylphenylsiloxane block, and/or
a block comprising fluorinated groups.

The film-forming polyurethanes as defined in the invention can also be obtained from polyesters, whether branched or unbranched, or alkyds comprising mobile hydrogen atoms which are modified by reaction with a diisocyanate and a bifunctional organic compound (for example dihydro, diamino or hydroxyamino) additionally comprising either a carboxylic acid or carboxylate group, or a sulphonic acid or sulphonate group, or also a neutralizable tertiary amine group or a quaternary ammonium group.

Mention may also be made, among film-forming polycondensates, of polyesters, polyesteramides, fatty-chain polyesters, polyamides and epoxy ester resins.

The polyesters can be obtained in a known way by polycondensation of dicarboxylic acid(s) with one or more polyols, in particular one or more diols.

The dicarboxylic acid or acids can be aliphatic, alicyclic or aromatic. Mention may be made, as examples of such acids, of oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norboranedicarboxylic acid, diglycolic acid, thiodipropionic acid, 2,5-naphthalene-dicarboxylic acid or 2,6-naphthalenedicarboxylic acid. These dicarboxylic acid monomers can be used alone or as a combination of at least two dicarboxylic acid monomers. The choice is in particular made, among these monomers, of phthalic acid, isophthalic acid and terephthalic acid.

The diol can be chosen from aliphatic, alicyclic and aromatic diols. Use is made in particular of a diol chosen from ethylene glycol, diethylene glycol, triethylene glycol, 1,3-propanediol, cyclohexanedi-methanol or 1,4-butanediol. Use may be made, as other polyols, of glycerol, pentaerythritol, sorbitol or trimethylolpropane.

The polyester amides can be obtained analogously to the polyesters by polycondensation of diacids with diamines or aminoalcohols. Use may be made, as diamine, of ethylenediamine, hexamethylenediamine, meta-phenylenediamine or para-phenylenediamine. Use may be made, as aminoalcohol, of monoethanolamine.

Use may also be made, in the present invention, of optionally modified polymers of natural origin, such as shellac resin, gum sandarac, dammars, elemis or copals, water-insoluble cellulose polymers, such as nitrocellulose and modified cellulose esters, such as carboxyalkylcellulose esters, for example those described in Patent Application US 2003/185774, and their blends.

Preferably, the film-forming polymer is chosen from water-insoluble cellulose polymers and from modified cellulose esters as defined above, and their blends.

The film-forming polymer or polymers are preferably present in an amount ranging from 10 to 40% by weight and better still from 15 to 30% by weight, with respect to the total weight of the composition.

The continuous phase can additionally comprise one or more oils and/or one or more organic solvents. Preferably, the continuous phase comprises at least one organic solvent and the composition is then provided in the form of a water-in-organic solvent emulsion.

Preferably, the organic solvent or solvents are only slightly soluble or insoluble in water.

The term “solvent which is only slightly soluble in water” is understood to mean, within the meaning of the present invention, a solvent having a solubility in water at 20° C. and at atmospheric pressure of less than 4%.

The term “solvent which is insoluble in water” is understood to mean, within the meaning of the present invention, a solvent having a solubility in water at 20° C. and at atmospheric pressure of less than 1% and preferably of less than 0.5%.

Mention may in particular be made, as examples of organic solvent, of:
ketones which are liquid at ambient temperature, such as methyl isobutyl ketone, diisobutyl ketone or isophorone;
short-chain esters comprising from 5 to 8 carbon atoms in total, such as propyl acetate, isopropyl acetate, n-butyl acetate, isopentyl acetate or methoxypropyl acetate;
ethers which are liquid at ambient temperature, such as dimethyl ether;
alkanes which are liquid at ambient temperature, such as decane, heptane, dodecane or cyclohexane;
aldehydes which are liquid at ambient temperature, such as benzaldehyde;
ethyl 3-ethoxypropionate;
carbonates, such as dimethyl carbonate; and
their mixtures.

Preferably, the organic solvent or solvents are chosen from short-chain esters comprising from 5 to 8 carbon atoms in total, such as propyl acetate, isopropyl acetate, n-butyl acetate, isopentyl acetate, methoxypropyl acetate, and their mixtures.

The organic solvent or solvents can be present in the composition according to the invention in an amount preferably ranging from 5 to 85% by weight and in particular from 25 to 80% by weight, with respect to the total weight of the composition.

Preferably, the nail varnish composition according to the invention is devoid of ethyl acetate.

The composition according to the invention can additionally comprise one or more plasticizing agents, for example in the continuous phase. Mention may in particular be made, as examples of plasticizing agent, of:
ethylene glycol derivatives, such as ethylene glycol or diethylene glycol ethers, for example diethylene glycol ethyl ether, diethylene glycol methyl ether, diethylene glycol butyl ether, diethylene glycol hexyl ether, ethylene glycol ethyl ether, ethylene glycol butyl ether or ethylene glycol hexyl ether;
polyol esters, such as propylene glycol diacetate;
propylene glycol derivatives, such as propylene glycol or dipropylene glycol ethers, in particular propylene glycol phenyl ether, dipropylene glycol butyl ether, tripropylene glycol butyl ether, propylene glycol methyl ether, dipropylene glycol ethyl ether, tripropylene glycol methyl ether or propylene glycol butyl ether;
esters of acids, in particular carboxylic acids, such as adipates, citrates, esters of tert-butyric acid, phthalates, sebacates, stearates, palmitates, carbonates, tartrates, benzoates, acetylricinoleates, glycolates or triacetates and more particularly diisobutyl adipate, diethyl adipate, triethyl citrate, tributyl citrate, triethyl acetylcitrate, tributyl acetylcitrate, triethylhexyl acetylcitrate, the ester of tert-butyric acid and of 2,2,4-trimethylpentane-1,3-diol, diethyl phthalate, dibutyl phthalate, dioctyl phthalate, dipentyl phthalate, dimethoxy-ethyl phthalate, butyl 2-ethylhexyl phthalate, dimethyl sebacate, dibutyl sebacate, ethyl stearate, 2-ethylhexyl palmitate, benzyl benzoate, butyl acetylricinoleate, glyceryl acetylricinoleate, butyl glycolate or glyceryl triacetate;
phosphates and more particularly tricresyl phosphate, tributyl phosphate, triphenyl phosphate or tributoxyethyl phosphate;
camphor, N-ethyl-o,p-toluenesulphonamide;
oxyethylenated derivatives, such as oxyethylenated oils, in particular vegetable oils, such as castor oil;
silicone oils; and
their mixtures.

Advantageously, the plasticizing agent is chosen from esters of acids such as described above and their mixtures and more particularly still from diisobutyl adipate, diethyl adipate, triethyl citrate, tributyl citrate, triethyl acetylcitrate, tributyl acetylcitrate, triethylhexyl acetylcitrate, the ester of tert-butyric acid and of 2,2,4-trimethylpentane-1,3-diol, and their mixtures.

The plasticizing agent or agents can be present in the composition according to the invention in an amount preferably ranging from 0.1 to 20% by weight and in particular from 0.5 to 10% by weight, with respect to the total weight of the composition.

The dispersed phase preferably comprises predominantly water, that is to say an amount of water preferably ranging from 40 to 99.99% by weight, better still from 60 to 99.99% by weight, with respect to the total weight of the dispersed phase.

With respect to the total weight of the composition according to the invention, the water is present in an amount preferably ranging from 15 to 90% by weight, better still from 20% to 70% by weight.

The composition according to the invention can additionally comprise at least one colouring material which can be chosen from pulverulent compounds and water-soluble or fat-soluble dyes. The colouring material can be present in an amount preferably ranging from 0.01 to 50% by weight, better still from 0.05 to 30% by weight and even better still from 0.1 to 20% by weight, with respect to the total weight of the composition.

The pulverulent compounds can be chosen from pigments and/or pearlescent agents and/or glitter normally used in nail varnishes.

The pigments can be white or coloured and inorganic and/or organic. Mention may be made, among inorganic pigments, of titanium dioxide, optionally surface-treated, zirconium or cerium oxides, and also iron or chromium oxides, manganese violet, ultramarine blue, chromium hydrate and ferric blue, or metal pigments, such as aluminium, copper or bronze. Mention may be made, among organic pigments, of carbon black, pigments of D & C type, lakes, based on cochineal carmine, of barium, strontium, calcium or aluminium, or guanine.

Mention may also be made of effect pigments, such as particles comprising an organic or inorganic and natural or synthetic substrate, for example, glass, acrylic, polyester, polyurethane or poly(ethylene terephthalate) resins, ceramics or aluminas, which may or may not be covered with metal substances, such as aluminium, gold, copper or bronze, or with metal oxides, such as titanium dioxide, iron oxide or chromium oxide, with inorganic or organic pigments, and their mixtures.

The term “effect pigments” is understood to mean pigments which confer a bulk colour different from the highlight colour, and it is possible to have several highlights.

The pearlescent agents can be chosen from white pearlescent pigments, such as mica covered with titanium oxide or with bismuth oxychloride, coloured pearlescent pigments, such as titanium oxide-coated mica with iron oxides, titanium oxide-coated mica with in particular ferric blue or chromium oxide or titanium oxide-coated mica with an organic pigment of the abovementioned type, and fluorophlogopite with iron oxides.

Use may also be made of pigments having goniochromatic properties, in particular liquid crystal properties, or mutilayer pigments.

The water-soluble dyes are, for example, beetroot juice and methylene blue.

Mention may in particular be made, as examples of fat-soluble dye, of Sudan red, DC Red 17, DC Green 6, β-carotene, soybean oil, Sudan brown, DC Yellow 11, DC Violet 2, DC Orange 5 and quinoline yellow.

The composition according to the invention can additionally comprise one or more fillers, in particular in a content ranging from 0.01 to 50% by weight, preferably from 0.01 to 30% by weight, with respect to the total weight of the composition. The term “fillers” should be understood as meaning colourless or white and inorganic or synthetic particles of any shape which are insoluble in the medium of the composition, whatever the temperature at which the composition is manufactured. These fillers serve in particular to modify the rheology or the texture of the composition.

The fillers can be inorganic or organic and of any shape, platelet, spherical or oblong, whatever the crystallographic form (for example, sheet, cubic, hexagonal, orthorhombic, and the like). Mention may be made of talc, mica, silica, kaolin, powders formed of polyamide (Nylon@)) (Orgasol® from Atochem), of poly-β-alanine and of polyethylene, powders formed of tetrafluoroethylene polymers (Teflon®), lauroyllysine, starch, boron nitride, hollow polymeric microspheres, such as those of poly(vinylidene chloride)/acrylonitrile, for example Expancel® (Nobel Industrie), or of acrylic acid copolymers (Polytrap® from Dow Corning), silicone resin microbeads (Tospearls® from Toshiba, for example), polyorganosiloxane elastomer particles, precipitated calcium carbonate, magnesium carbonate, basic magnesium carbonate, hydroxyapatite, hollow silica microspheres (Silica Beads® from Maprecos), glass or ceramic micro-capsules, or metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

The composition according to the invention can additionally comprise at least one additive commonly used in cosmetic compositions and known to a person skilled in the art as being capable of being incorporated in a nail varnish composition.

These additives can be chosen from coalescence agents, thickening agents, preservatives, fragrances, oils, waxes, surfactants, antioxidants, agents for combating free radicals, spreading agents, wetting agents, dispersing agents, antifoaming agents, neutralizing agents, stabilizers, active principles chosen from essential oils, organic and inorganic UV/sun screening agents, fragrances, moisturizing agents, vitamins, proteins, ceramides, plant extracts, and the like, and their mixtures.

Mention may in particular be made, as examples of thickening agent, of hydrophilic clays, such as hectorites, or bentonites, such as, for example, Laponite XLS, supplied by Rockwood, associative thickening agents other than the gelling agents described above, such as associative polyurethanes, for example Serad FX, sold by Servo, water-soluble cellulose thickening agents, such as hydroxyethyl-cellulose, or natural gums, such as xanthan gum.

Preferably, the nail varnish composition according to the invention is devoid of hydrophilic clay. Preferably, the nail varnish composition according to the invention is devoid of bentonite.

Of course, a person skilled in the art will take care to choose this or these optional additional compounds and/or their amounts so that the properties of the composition according to the invention are not, or not substantially, detrimentally affected by the envisaged addition.

The composition as defined above, in the emulsion form, can be prepared in the following way:
by first preparing an aqueous gel by mixing water, one or more gelling agents and optionally at least one water-soluble active principle;
by preparing the continuous phase by mixing the organic solvent or solvents and optionally at least one other compound, such as, for example, a film-forming polymer, a plasticizing agent or a dye;
by introducing the aqueous gel into the continuous phase with vigorous stirring in order to produce the emulsion.

The invention also relates to a method for making up the nails comprising application, to the nails, of at least one layer of a nail varnish composition as defined above.

Another subject-matter is a cosmetic article comprising:
i) a container comprising a nail varnish composition as defined above; and
ii) an applicator for withdrawing the product present in the container and applying it to the nails, the applicator being in the form of a brush, of a spatula, of a foam tip or of a flexible strip defining an applicator face.

The container can be in any appropriate form. It can in particular be in the form of a bottle and can be at least partially made of a material such as glass. However, materials other than glass can be used, such as thermoplastics, for example polypropylenes (PP) or polyethylenes (PE), or such as a metal.

The container is equipped with an applicator which can be in the form of a brush composed of at least one tuft of hairs. Alternatively, the applicator is provided in a form other than a brush, for example in the form of a spatula or of a foam tip or of a flexible strip defining an applicator face.

The example which follows illustrates the invention without implied limitation. Unless otherwise indicated, the amounts shown are expressed in grams.

### EXAMPLE

The nail varnish composition was prepared from the ingredients shown in the following table:

| | |
|---|---|
| Acrylamide/sodium acrylamido-2-methyl- | 0.2 |
| propanesulphonate copolymer as a 40% by weight | |
| inverse emulsion in isoparaffin/ethanol⁽¹⁾ | |
| Oxyethylenated polydimethylsiloxane (PDMS) having | 0.5 |
| methoxy endings (viscosity: 18-28 cSt) | |
| D&C Red 7 | 1.59 |
| D&C Red 6 | 1.59 |
| N-Ethyl-o,p-toluenesulphonamide | 4.24 |
| Tributyl acetylcitrate | 5.30 |
| Deionized water | 19.8 |
| Nitrocellulose at 30% by weight in isopropyl | 20.03 |
| alcohol | |
| Butyl acetate q.s for | 100 |

⁽¹⁾sold under the name Sepigel 305 by SEPPIC

First of all, the aqueous gel is prepared by mixing the water with the acrylamide/sodium acrylamido-2-methylpropanesulphonate copolymer.

The continuous phase is subsequently prepared by mixing the butyl acetate, the film-forming polymer, the plasticizing agents, the dyes and the oxyethylenated PDMS having methoxy endings. The aqueous gel is then added to the continuous phase with vigorous stirring in order to obtain the water-in-organic solvent emulsion.

The varnish composition obtained after mixing the above ingredients is stable and fluid.

The varnish is easily applied and results, after drying, in a homogeneous, glossy and flexible film which adheres well to the nail.

The above written description of the invention provides a manner and process of making and using it such that any person skilled in this art is enabled to make and use the same, this enablement being provided in particular for the subject matter of the appended claims, which make up a part of the original description and including a nail varnish composition in the form of an emulsion comprising at least one continuous phase and at least one dispersed phase and one or more water-soluble or water-dispersible polymers obtained from acrylamido-2-methylpropanesulphonic acid (or AMPS) or from one of its salts and from water-soluble monomers having ethylenic unsaturation(s) as gelling agent.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed. As used herein, the words “a” and “an” and the like carry the meaning of “one or more.”

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A nail varnish composition in the form of an emulsion comprising at least one continuous phase and at least one dispersed phase and one or more water-soluble or water-dispersible polymers obtained from acrylamido-2-methylpropanesulphonic acid (or AMPS) or from one of its salts and from water-soluble monomers having ethylenic unsaturation(s) as gelling agent.

2. The nail varnish composition according to claim 1, wherein the continuous phase predominantly comprises one or more oils or one or more organic solvents and the dispersed phase is aqueous.

3. The nail varnish composition according to claim 1, wherein the continuous phase of the emulsion is present in an amount ranging from 10 to 85% by weight with respect to the total weight of the composition.

4. The nail varnish composition according to claim 1, wherein the dispersed phase of the emulsion is present in an amount ranging from 15 to 90% by weight with respect to the total weight of the composition.

5. The nail varnish composition according to claim 1, wherein the continuous phase and the dispersed phase of the emulsion are present in a ratio by weight ranging from 1/10 to 6.

6. The nail varnish composition according to claim 1, wherein the gelling agent is chosen from copolymers obtained from AMPS or from one of its salts and from acrylamide or methacrylamide, copolymers of AMPS or of one of its salts and of vinylpyrrolidone or of vinylformamide, copolymers of AMPS or one of its salts and of sodium acrylate, or copolymers of AMPS or of one of its salts and of hydroxyethyl acrylate.

7. The nail varnish composition according to claim 1, wherein the gelling agent or agents are present in an amount ranging from 0.01 to 10% by weight with respect to the total weight of the composition.

8. The nail varnish composition according to claim 2, wherein the continuous phase predominantly comprises one or more organic solvents chosen from ketones which are liquid at ambient temperature, short-chain esters comprising from 5 to 8 carbon atoms in total, ethers which are liquid at ambient temperature, alkanes which are liquid at ambient temperature, aldehydes which are liquid at ambient temperature, ethyl 3-ethoxypropionate, carbonates and their mixtures.

9. The nail varnish composition according to claim 8, wherein the organic solvent or solvents are chosen from methyl isobutyl ketone, diisobutyl ketone, isophorone, propyl acetate, isopropyl acetate, n-butyl acetate, isopentyl acetate, methoxypropyl acetate, dimethyl ether, decane, heptane, dodecane, cyclohexane, benzaldehyde, ethyl 3-ethoxypropionate, dimethyl carbonate and their mixtures.

10. The nail varnish composition according to claim 1, wherein it further comprises one or more film-forming polymers.

11. The nail varnish composition according to claim 10, wherein the film-forming polymer is chosen from synthetic polymers obtained by the radical route or by polycondensation, polymers of natural origin and their mixtures.

12. The nail varnish composition according to claim 10, comprising one or more acrylic and/or vinyl homopolymers or copolymers.

13. The nail varnish composition according to claim 10, comprising one or more film-forming polymers chosen from polyurethanes which are anionic, cationic, nonionic or amphoteric, polyurethane-acrylics, polyurethane-polyvinylpyrrolidones, polyester-polyurethanes, polyether-polyurethanes, polyureas, polyurea/polyurethanes, silicone polyurethanes, and their mixtures.

14. The nail varnish composition according to claim 10, comprising one or more film-forming polymers chosen from shellac resin, gum sandarac, dammars, elemis, copals, water-insoluble cellulose polymers and their mixtures.

15. The nail varnish composition according to claim 10, comprising one or more film-forming polymers chosen from nitrocellulose and modified cellulose esters.

16. The nail varnish composition according to claim 10, wherein the film-forming polymer is present in the continuous phase.

17. The nail varnish composition according to claim 10, wherein the film-forming polymer or polymers are present in an amount ranging from 10 to 40% by weight with respect to the total weight of the composition.

18. The nail varnish composition according to claim 1, wherein it further comprises one or more plasticizing agents.

19. The nail varnish composition according to claim 18, wherein the plasticizing agent is chosen from:
ethylene glycol derivatives;
polyol esters;
propylene glycol derivatives;
esters of acids;
phosphates;
camphor, N-ethyl-o,p-toluenesulphonamide;
oxyethylenated derivatives;
silicone oils; and
their mixtures.

20. The nail varnish composition according to claim 19, wherein the plasticizing agent is chosen from:
diethylene glycol ethyl ether, diethylene glycol methyl ether, diethylene glycol butyl ether, diethylene glycol hexyl ether, ethylene glycol ethyl ether, ethylene glycol butyl ether or ethylene glycol hexyl ether;
propylene glycol diacetate;
propylene glycol phenyl ether, dipropylene glycol butyl ether, tripropylene glycol butyl ether, propylene glycol methyl ether, dipropylene glycol ethyl ether, tripropylene glycol methyl ether or propylene glycol butyl ether;
diisobutyl adipate, diethyl adipate, triethyl citrate, tributyl citrate, triethyl acetylcitrate, tributyl acetylcitrate, triethylhexyl acetyl-citrate, the ester of tert-butyric acid and of 2,2,4-trimethylpentane-1,3-diol, diethyl phthalate, dibutyl phthalate, dioctyl phthalate, dipentyl phthalate, dimethoxyethyl phthalate, butyl 2-ethylhexyl phthalate, dimethyl sebacate, dibutyl sebacate, ethyl stearate, 2-ethylhexyl palmitate, benzyl benzoate, butyl acetyl-ricinoleate, glyceryl acetylricinoleate, butyl glycolate or glyceryl triacetate;
tricresyl phosphate, tributyl phosphate, triphenyl phosphate or tributoxyethyl phosphate;
camphor, N-ethyl-o,p-toluenesulphonamide;
oxyethylenated oils;
silicone oils; and
their mixtures.

21. The nail varnish composition according to claim 19, wherein the plasticizing agent or agents are present in an amount ranging from 0.1 to 20% by weight, with respect to the total weight of the composition.

22. The nail varnish composition according to claim 10, wherein the dispersed phase predominantly comprises water.

23. The nail varnish composition according to claim 22, wherein the dispersed phase comprises from 40 to 99.99% by weight of water, with respect to the total weight of the dispersed phase.

24. The nail varnish composition according to claim 1, further comprising at least one colouring material.

25. The nail varnish composition according to claim 24, wherein the colouring material is present in an amount ranging from 0.01 to 50% by weight, with respect to the total weight of the composition.

26. A method for making up a nail, comprising the application, to the nail, of at least one layer of a nail varnish composition according to claim 1.

27. A cosmetic article, comprising:
(i) a container, and, therein a nail varnish according to claim 1, and
(ii) an applicator for withdrawing the nail varnish present in the container and applying it to a nail, the applicator being in the form of a brush, of a spatula, of a foam tip or of a flexible strip defining an applicator face.
